(19) United States
(12) Patent Application Publication
(10) Pub. No.: US 2023/0303697 A1
(43) Pub. Date: Sep. 28, 2023

(54) Antiaging effect of Intradermal Administration of Immune Checkpoint Inhibitors

(71) Applicant: MYKARA, INC, Redondo Beach, CA (US)

(72) Inventors: Sarah Tomassetti, Redondo Beach, CA (US); Mykola Onyshchenko, Los Angeles, CA (US)

(73) Assignee: MYKARA, INC., Redondo Beach, CA (US)

(21) Appl. No.: 17/803,947

(22) Filed: Jan. 31, 2023

Related U.S. Application Data
(60) Provisional application No. 63/361,958, filed on Feb. 2, 2022.

(51) Int. Cl. C07K 16/28; A61P 17/00

(52) U.S. Cl. CPC C07K 16/2827; A61P 17/00; C07K 16/2818

(57) ABSTRACT

The present invention is generally directed to compositions and methods for preventing, ameliorating or treating aging effects. In one aspect, the present invention is directed to a method of treating an effect of aging. The effect of aging is selected from a group consisting of loss of hair pigmentation, hair loss, increase in fine lines, increase in skin laxity, increase of skin sun spots, loss of natural skin color, loss of skin elasticity, increase in skin pore size and loss of skin hydration. The method comprises the steps of: obtaining or preparing an Immune Checkpoint Inhibitor formulation, wherein the formulation comprises an Immune Checkpoint Inhibitor and water, and wherein the concentration of the Immune Checkpoint Inhibitor in the formation ranges from 1 mg/mL to 60 mg/mL; intradermally, topically or electrophoretically administering a portion of the formula to a patient experiencing an aging effect to a skin area on the patient, wherein the amount of Immune Checkpoint Inhibitor administered in the formulation ranges from 1 mg to 750 mg, wherein an aging effect is lessened by a percentage ranging from 1 percent to 50 percent within three weeks after intradermal administration.

### CROSS-REFERENCE TO RELATED APPLICATION

This application claims the benefit of U.S. Provisional Application No. 63/361,958, filed Feb. 2, 2022, which is incorporated herein in its entirety.

### FIELD OF THE INVENTION

The present invention is generally directed to compositions and methods for preventing, ameliorating or treating aging effects.

### BACKGROUND OF THE INVENTION

There have been reports on the use of Immune Checkpoint Inhibitors. Sibaud, for example, discusses the following: “The development of immune checkpoint inhibitors [monoclonal antibodies targeting cytotoxic T lymphocyte-associated antigen-4 (CTLA-4), programmed cell death protein 1 (PD-1) or programmed death ligand 1 (PD-L1)] represents a major breakthrough in cancer therapy. Although they present a favorable risk/benefit ratio, immune checkpoint blockade therapies have a very specific safety profile. Due to their unique mechanism of action, they entail a new spectrum of adverse events that are mostly immune related [immune-related adverse events (irAEs)], notably mediated by the triggering of cytotoxic CD4+/CD8+ T cell activation. Cutaneous toxicities appear to be one of the most prevalent irAEs, both with anti-PD-1 and anti-CTLA-4 agents or with the newly developed anti-PD-L1 agents, which corresponds to a class effect. They are observed in more than one-third of the treated patients, mainly in the form of a maculopapular rash (eczema-like spongiotic dermatitis) and pruritus. A wide range of other dermatologic manifestations can also occur, including lichenoid reactions, psoriasis, acneiform rashes, vitiligo-like lesions, autoimmune skin diseases (e.g., bullous pemphigoid, dermatomyositis, alopecia areata), sarcoidosis or nail and oral mucosal changes. In addition, the use of anti-CTLA-4 and anti-PD-1 therapies in combination is associated with the development of more frequent, more severe and earlier cutaneous irAEs compared to single agents. In most cases, these dysimmune dermatologic adverse events remain self-limiting and readily manageable. Early recognition and adequate management, however, are critical to prevent exacerbation of the lesions, to limit treatment interruption and to minimize quality of life impairment. This review describes the variable clinical and histopathologic aspects of dermatologic irAEs induced by immune checkpoint inhibitors. Appropriate treatment and counseling are also proposed, with a step-by-step approach for optimized management by both practicing oncologists and dermatologists.” Am J Clin Dermatol. 2018 Jun;19(3):345-361, Abstract.

Darvin et al., report the following regarding Immune Checkpoint Inhibitors: “Cancer” growth and progression are associated with immune suppression. Cancer cells have the ability to activate different immune checkpoint pathways that harbor immunosuppressive functions. Monoclonal antibodies that target immune checkpoints provided an immense breakthrough in cancer therapeutics. Among the immune checkpoint inhibitors, PD-1/PD-L1 and CTLA-4 inhibitors showed promising therapeutic outcomes, and some have been approved for certain cancer treatments, while others are under clinical trials. Recent reports have shown that patients with various malignancies benefit from immune checkpoint inhibitor treatment. However, mainstream initiation of immune checkpoint therapy to treat cancers is obstructed by the low response rate and immune-related adverse events in some cancer patients. This has given rise to the need for developing sets of biomarkers that predict the response to immune checkpoint blockade and immune-related adverse events. In this review, we discuss different predictive biomarkers for anti-PD-1/PD-L1 and anti-CTLA-4 inhibitors, including immune cells, PD-L1 overexpression, neoantigens, and genetic and epigenetic signatures. Potential approaches for further developing highly reliable predictive biomarkers should facilitate patient selection for and decision-making related to immune checkpoint inhibitor-based therapies.” Experimental & Molecular Medicine, Vol. 50, pages 1-11 (2018).

Franzin et al. review the following: “Immune checkpoint inhibitors (ICIs) are a novel class of immunotherapy drugs that have improved the treatment of a broad spectrum of cancers as metastatic melanoma, non-small lung cancer or renal cell carcinoma. These humanized monoclonal antibodies target inhibitory receptors (e.g. CTLA-4, PD-1, LAG-3, TIM-3) and ligands (PD-L1) expressed on T lymphocytes, antigen presenting cells and tumor cells and elicit an anti-tumor response by stimulating immune system. Nevertheless, the improved overall survival is complicated by the manifestation of Immune-related Adverse Effects (irAEs). During treatment with ICIs, the most common adverse kidney effect is represented by the development of acute kidney injury (AKI) with the acute tubulointerstitial nephritis as recurrent histological feature. The mechanisms involved in ICIs-induced AKI include the re-activation of effector T cells previously stimulated by nephrotoxic drugs (i.e. by antibiotics), the loss of tolerance versus self-renal antigens, the increased PD-L1 expression by tubular cells or the establishment of a pro-inflammatory milieu with the release of self-reactive antibodies. For renal transplant recipient treated with ICIs, the increased incidence of rejection is a serious concern. Therefore, the combination of ICIs with mTOR inhibitors represents an emerging strategy. Finally, it is relevant to anticipate which patients under ICIs would experience severe irAEs and from a kidney perspective, to predict patients with higher risk of AKI. Here, we provide a detailed overview of ICIs-related nephrotoxicity and the recently described multicenter studies. Several factors have been reported as biomarkers of ICIs-irAEs, in this review we speculate on potential biomarkers for ICIs-associated AKI.” Front. Immunol., 08 Oct. 2020.

Despite the various reports and discussions related to Immune Checkpoint Inhibitors, there is still a need for novel compositions and methods regarding them.

### SUMMARY OF THE INVENTION

In one aspect, the present invention is directed to a method of treating an effect of aging. The effect of aging is selected from a group consisting of loss of hair pigmentation, hair loss, increase in fine lines, increase in,skin laxity, increase of skin sun spots, loss of natural skin color, loss of skin elasticity, increase in skin pore size and loss of skin hydration. The method comprises the steps of: obtaining or preparing an Immune Checkpoint Inhibitor formulation, wherein the formulation comprises an Immune Checkpoint Inhibitor and a solvent (e.g., water), and wherein the concentration of the Immune Checkpoint Inhibitor in the formulation ranges from 1 mg/mL to 60 mg/mL; administering a portion of the formula to a patient experiencing an aging effect to a skin area and/or skin derivatives (e.g., hair) on the patient - typically through intradermal administration, topical administration or electrophoretic administration - wherein the amount of Immune Checkpoint Inhibitor administered in the formulation typically ranges from 1 mg to 750 mg, wherein an aging effect is lessened by a percentage ranging from 1 percent to 100 percent (e.g., 1 percent to 50 percent) within six months (e.g., three weeks or three months) after administration.

### DETAILED DESCRIPTION OF THE INVENTION

“Immune Checkpoint Inhibitor” refers to a compound, typically a monoclonal antibody, that “blocks proteins called immune checkpoints that are made by some types of immune system cells, such as T cells, and some cancer cells. These checkpoints help keep immune responses from being too strong and sometimes can keep T cells from killing cancer and non-cancer cells. When these checkpoints are blocked, T cells can kill cancer cells better. Examples of checkpoint proteins found on T cells or cancer cells include PD-1/PD-L1 and CTLA-4/B7-1/B7-2.” See, https://www.cancer.gov/publications/dictionaries/cancer-terms/def/immune-checkpoint-inhibitor. Nonlimiting examples of Immune Checkpoint Inhibitors include: Pembrolizumab (PD-1); Nivolumab (PD-1); Cemiplimab (PD-1); Atezolizumab (PD-L1); Avelumab (PD-L1); Durvalumab (PD-L1); Ipilimumab (CTLA-4); Tremelimumab (CTLA-4), and others in development. Nonlimiting examples of other Immune Checkpoint Inhibitors under development include: LAG525 (IMP701); REGN3767 (R3767); BI 754,091; Tebotelimab (MGD013); FS118; MBG453; Sym023; TSR-022; MGC018; FPA150; EOS100850; AB928; CPI-006; Monalizumab; COM701; Defactinib; PF-04136309; MSC-1; Hu5F9-G4 (5F9); ALX148; TTI-662; RRx-001; Lacnotuzumab (MCS110); Emactuzumab (RG7155); Pexidartinib (PLX3397); CAN04; Canakinumab (ACZ885); BMS-986253; Pepinemab (VX15/2503); Trebananib; FP-1305. See, Marin-Acevedo et al. “Next generation of immune checkpoint inhibitors and beyond” Journal of Hematology & Oncology volume 14, Article number: 45 (2021), and references therein.

“Intradermal administration” refers to administration into a patient’s dermis, just below the epidermis. Administration is typically by injection, i.e., intradermal injection (“ID”).

“Topical administration” refers to application to body surfaces such as the skin or mucous membranes. In certain cases, topical medications are applied directly to the skin.

“Electrophoretic administration” refers to transdermal drug delivery using a voltage gradient on the skin.

“Antiaging Effect” refers to an effect that stops, slows or reverses physiological processes typically associated with aging. Nonlimiting examples of antiaging effects include: hair repigmentation, hair regrowth, fine line reduction in skin, decrease in skin laxity, reduction of skin sun spots, restoration of natural skin color, skin resurfacing, reduction in size of skin pores, improvement of skin hydration.

Hair repigmentation can be measured, for example, using a digital camera such as a Panasonic DC-FZ80 Digital Camera. Microscopic imaging of hair follicles, individual hair shafts and root ends can be performed, for instance, using an Olympus BX61 upright microscope. One can measure repigmentation by imaging hair prior to treatment and then again after treatment. Percentage of repigmentation can be determined by comparing the image prior to treatment to the image post treatment. See, for example, eLife 2021;10:e67437.

Hair regrowth can be measured, for example, using optical microscopy and digital analysis. This method allows one to measure parameters such as the number of hairs per unit area and hair diameter. Percentage of hair regrowth can be measured by comparing the number of hairs per unit area prior to treatment and post treatment of a similar comparison of hair diameter. See, for example, Hayashi et al. Br. J. Dermatol. 1991 Aug;125(2):123-9.

Fine line reduction in skin can be measured, for example, using a profilometric method, where wrinkle size and function are determined. Parameters such as Wrinkle Depth, Wrinkle Area and Wrinkle Volume can be measured. Percentage of fine line reduction can be determined by comparing, for instance, Wrinkle Depth, Wrinkle Area and Wrinkle Volume prior to treatment and post treatment. See, Hatzis, Micron. 2004;35(3):201-19.

Decrease in skin laxity can be measured, for example, using a Cutometer SEM 575 skin elasticity meter. Percentage of decrease in skin laxity can be determined by comparing, for instance, the elasticity of skin prior to treatment and post treatment. See, Koch et al. Arch. Facial Plast. Surg. Oct-Dec 1999;1(4):272-5.

Reduction of skin sun spots can be measured, for example, using a digital camera, similar to that discussed for measurement of hair repigmentation. Percentage of sun spot decrease or size reduction can be determined by comparing, for instance, the number and size of sun spots prior to treatment and post treatment.

Restoration of natural skin color can be measured, for example, using a spectrophotometric measurement of skin color. Percentage of restoration of natural skin color can be determined by taking spectrophotometric measurements of skin prior to treatment and post treatment. See, Wang et al. Color and Research Application, Vol. 42, Issue 6, December 2017, pages 764-774.

Skin resurfacing success can be measured, for example, similarly to decrease in skin laxity. A Cutometer SEM 575 skin elasticity meter can be used to determine an increase in skin elasticity by measuring elasticity of skin prior to treatment and post treatment. See, Koch et al. Arch. Facial Plast. Surg. Oct-December 1999;1(4):272-5.

Reduction in size of skin pores can be measured, for example, using a digital camera. Percentage of size reduction can be determined by comparing, for instance, the number and size of skin pores prior to treatment and post treatment. See, Kotkin, et al. Journal of the American Academy of Dermatology, Vol. 50, Issue 3, Supplement, P32, Mar. 1, 2004.

Improvement of skin hydration can be measured, for example, by using an infrared optical spectroscopic set-up. Detection at various wavelengths - e.g., 1720 nm, 1750 nm and 1770 nm - can be performed. Percentage of improvement of skin hydration can be determined by comparing, for instance, spectroscopic data of skin prior to treatment and post treatment. See, Ezerskaia, et al. Biomed Opt Express. 2016 Jun 1; 7(6): 2311-2320.

Pembrolizumab is an antibody used in cancer immunotherapy that treats melanoma, lung cancer, head and neck cancer, Hodgkin lymphoma, and stomach cancer. It is sold under the brand name Keytruda and has also been referred to as MK-3475 and lambrolizumab. A typical dose of Pembrolizumab for treating cancer is 200 mg every three weeks given intravenously.

Nivolumab is an antibody used to treat a number of cancer types. It is sold under the brand name Opdivo and has also been referred to as ONO-4538, BMS-936558 and MDX1106. A typical dose of Nivolumab for treating cancer is 360 mg every three weeks given intravenously.

Cemiplimab is an antibody used to treat squamous cell skin cancer. It is sold under the brand name Libtayo and has also been referred to as REGN-2810, REGN2810 and cemiplimabrwlc. A typical dose of Cemiplimab for treating cancer is 350 mg every three weeks given intravenously.

Atezolizumab is an antibody used to treat urothelial carcinoma, non-small cell lung cancer, triple-negative breast cancer, small cell lung cancer, and hepatocellular carcinoma. It is sold under the brand name Tecentriq and has also been referred to as MPDL3280A and RG7446. A typical dose of Atezolizumab for treating cancer is 1200 mg every three weeks given intravenously.

Avelumab is an antibody used to treat Merkel cell carcinoma, urothelial carcinoma, and renal cell carcinoma. It is sold under the brand name Bavencio and has also been referred to as MSB0010718C. A typical dose of Atezolizumab for treating cancer is 800 mg every three weeks given intravenously.

Durvalumab is an antibody that has been FDA-approved for the treatment of cancer. It has also been referred to as MEDI4736 and MEDI-4736. A typical dose of Durvalumab for treating cancer is 1500 mg every three weeks given intravenously.

Ipilimumab is an antibody that is used to treat certain forms of cancer. It is sold under the brand name Yervoy and has also been referred to as BMS-734016, MDX-010 and MDX-101. A typical dose of Ipilimumab for treating cancer is 1-3 mg/kg every three weeks given intravenously.

Tremelimumab is an antibody being developed for the treatment of certain cancer forms. It has also been referred to as ticilimumab and CP-675,206. A typical dose of Tremelimumab for treating cancer is 750 mg every four weeks given intravenously.

Current antiaging therapies for skin and hair are achieved by using local administration of certain medications (e.g., Minoxidil), vitamins, micronutrients, fillers, growth factors, autologous plasma with creams, lotions, serums with or without injecting devices. Other antiaging methods include non-medicated microneedling and use of physical factors (e.g., light therapy with or without lasers, heat/cold therapies, ultrasound, electromagnetic waves, etc.).

The present invention is directed to administration of a Checkpoint Inhibitor, or a combination of one or more Immune Checkpoint inhibitors, to skin areas or skin derivative areas on a patient to prevent, ameliorate or treat the effects of aging (e.g., loss of hair pigmentation, hair loss, increase in fine skin lines, increase in skin laxity, increase of skin sun spots, loss of natural skin color, loss of skin elasticity, increase in size of skin pores, loss of skin hydration), where the administration is typically intradermal administration, topical administration or electrophoretic administration. Typically, a skin area or skin derivative area experiencing an aging effect — or that could experience an aging effect — is identified. An Immune Checkpoint Inhibitor is formulated (with solvent, e.g. Normal Saline), and either intradermally administered using a microneedling device or by injection using a single-needle syringe or administered by other means, including topical and electrophoretic administration. The number of treatments/administrations of a Checkpoint Inhibitor to a patient will vary - e.g., one treatment to ten treatments, depending upon progress monitored by a physician.

The following are non-limiting examples of compositions according to the present invention:

1. An Immune Checkpoint Inhibitor in Normal Saline at a concentration ranging from 1 mg/mL to 60 mg/mL, 1 mg/mL to 30 mg/mL, 1 mg/mL to 10 mg/mL or 1 mg/mL to 5 mg/mL for the treatment of aging effects (anti-aging treatment or prevention) in a patient.

2. Pembrolizumab in Normal Saline at a concentration ranging from 1 mg/mL to 25 mg/mL, 1 mg/mL to 15 mg/mL, 1 mg/mL to 5 mg/mL or 1 mg/mL to 2 mg/mL for the treatment of aging effects (anti-aging treatment or prevention) in a patient.

3. Nivolumab in Normal Saline at a concentration ranging from 1 mg/mL to 10 mg/mL, 1 mg/mL to 7 mg/mL, 1 mg/mL to 5 mg/mL or 1 mg/mL to 2 mg/mL for the treatment of aging effects (anti-aging treatment or prevention) in a patient.

4. Cemiplimab in Normal Saline at a concentration ranging from 1 mg/mL to 50 mg/mL, 1 mg/mL to 25 mg/mL, 1 mg/mL to 10 mg/mL or 1 mg/mL to 5 mg/mL for the treatment of aging effects (anti-aging treatment or prevention) in a patient.

5. Atezolizumab in Normal Saline at a concentration ranging from 1 mg/mL to 60 mg/mL, 1 mg/mL to 30 mg/mL, 1 mg/mL to 10 mg/mL or 1 mg/mL to 5 mg/mL for the treatment of aging effects (anti-aging treatment or prevention) in a patient.

6. Avelumab in Normal Saline at a concentration ranging from 1 mg/mL to 20 mg/mL, 1 mg/mL to 10 mg/mL, 1 mg/mL to 5 mg/mL or 1 mg/mL to 2 mg/mL for the treatment of aging effects (anti-aging treatment or prevention) in a patient.

7. Durvalumab in Normal Saline at a concentration ranging from 1 mg/mL to 50 mg/mL, 1 mg/mL to 25 mg/mL, 1 mg/mL to 10 mg/mL or 1 mg/mL to 5 mg/mL for the treatment of aging effects (anti-aging treatment or prevention) in a patient.

8. Ipilimumab in Normal Saline at a concentration ranging from 1 mg/mL to 5 mg/mL, 1 mg/mL to 4 mg/mL, 1 mg/mL to 3 mg/mL or 1 mg/mL to 2 mg/mL for the treatment of aging effects (anti-aging treatment or prevention) in a patient.

9. Tremelimumab in Normal Saline at a concentration ranging from 1 mg/mL to 5 mg/mL, 1 mg/mL to 4 mg/mL, 1 mg/mL to 3 mg/mL or 1 mg/mL to 2 mg/mL for the treatment of aging effects (anti-aging treatment or prevention) in a patient.

The following are non-limiting examples of methods according to the present invention:

1. Intradermal, topical or electrophoretic administration of an Immune Checkpoint Inhibitor at a dose ranging from 1 mg to 750 mg for the treatment of hair pigmentation loss, where the treatment results in a percentage of hair repigmentation ranging from 1 percent to 100 percent, from 1 to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

2. Intradermal, topical or electrophoretic administration of an Immune Checkpoint Inhibitor at a dose ranging from 1 mg to 750 mg for the treatment of hair loss, wherein the treatment results in a percentage of hair regrowth ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

3. Intradermal, topical or electrophoretic administration of an Immune Checkpoint Inhibitor at a dose ranging from 1 mg to 750 mg for the treatment of skin fine lines (e.g., reduction of skin fine lines), wherein the treatment results in a percentage of skin fine line reduction ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

4. Intradermal, topical or electrophoretic administration of an Immune Checkpoint Inhibitor at a dose ranging from 1 mg to 750 mg for the treatment of skin laxity, wherein the treatment results in a percentage reduction of skin laxity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

5. Intradermal, topical or electrophoretic administration of an Immune Checkpoint Inhibitor at a dose ranging from 1 mg to 750 mg for the treatment of skin sun spots, wherein the treatment results in a percentage reduction of the number of skin sun spots ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

6. Intradermal, topical or electrophoretic administration of an Immune Checkpoint Inhibitor at a dose ranging from 1 mg to 750 mg for the treatment of skin color, wherein the treatment results in a percentage restoration of natural skin color ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

7. Intradermal, topical or electrophoretic administration of an Immune Checkpoint Inhibitor at a dose ranging from 1 mg to 750 mg for the treatment of skin elasticity, wherein the treatment results in a percentage increase of skin elasticity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

8. Intradermal, topical or electrophoretic administration of an Immune Checkpoint Inhibitor at a dose ranging from 1 mg to 750 mg for the treatment of skin pores, wherein the treatment results in a percentage decrease of skin pore size ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

9. Intradermal, topical or electrophoretic administration of an Immune Checkpoint Inhibitor at a dose ranging from 1 mg to 750 mg for the treatment of skin hydration, wherein the treatment results in a percentage increase of skin hydration ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or, three months after the treatment).

10. Intradermal, topical or electrophoretic administration of Pembrolizumab at a dose ranging from 1 mg to 100 mg, 1 mg to 50 mg, 1 mg to 25 mg or 1 mg to 10 mg for the treatment of hair pigmentation loss, where the treatment results in a percentage of hair repigmentation ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

11. Intradermal, topical or electrophoretic administration of Pembrolizumab at a dose ranging from 1 mg to 100 mg, 1 mg to 50 mg, 1 mg to 25 mg or 1 mg to 10 mg for the treatment of hair loss, wherein the treatment results in a percentage of hair regrowth ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

12. Intradermal, topical or electrophoretic administration of Pembrolizumab at a dose ranging from 1 mg to 100 mg, 1 mg to 50 mg, 1 mg to 25 mg or 1 mg to 10 mg for the treatment of skin fine lines (e.g., reduction of skin fine lines), wherein the treatment results in a percentage of skin fine line reduction ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

13. Intradermal, topical or electrophoretic administration of Pembrolizumab at a dose ranging from 1 mg to 100 mg, 1 mg to 50 mg, 1 mg to 25 mg or 1 mg to 10 mg for the treatment of skin laxity, wherein the treatment results in a percentage reduction of skin laxity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

14. Intradermal, topical or electrophoretic administration of Pembrolizumab at a dose ranging from 1 mg to 100 mg, 1 mg to 50 mg, 1 mg to 25 mg or 1 mg to 10 mg for the treatment of skin sun spots, wherein the treatment results in a percentage reduction of the number of skin sun spots ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

15. Intradermal, topical or electrophoretic administration at a dose ranging from 1 mg to 100 mg, 1 mg to 50 mg, 1 mg to 25 mg or 1 mg to 10 mg for the treatment of skin color, wherein the treatment results in a percentage restoration of natural skin color ranging from 1 to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

16. Intradermal, topical or electrophoretic administration of Pembrolizumab at a dose ranging from 1 mg to 100 mg, 1 mg to 50 mg, 1 mg to 25 mg or 1 mg to 10 mg for the treatment of skin elasticity, wherein the treatment results in a percentage increase of skin elasticity ranging from 1 to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

17. Intradermal, topical or electrophoretic administration of Pembrolizumab at a dose ranging from 1 mg to 100 mg, 1 mg to 50 mg, 1 mg to 25 mg or 1 mg to 10 mg for the treatment of skin pores, wherein the treatment results in a percentage decrease of skin pore size ranging from 1 to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

18. Intradermal, topical or electrophoretic administration of Pembrolizumab at a dose ranging from 1 mg to 100 mg, 1 mg to 50 mg, 1 mg to 25 mg or 1 mg to 10 mg for the treatment of skin hydration, wherein the treatment results in a percentage increase of skin hydration ranging from 1 percent to 100 percent from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks of three months after the treatment).

19. Intradermal, topical or electrophoretic administration of Nivolumab at a dose ranging from 1 mg to 180 mg, 1 mg to 90 mg, 1 mg to 45 mg or 1 mg to 18 mg for the treatment of hair pigmentation loss, where the treatment results in a percentage of hair repigmentation ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

20. Intradermal, topical or electrophoretic administration of Nivolumab at a dose ranging from 1 mg to 180 mg, 1 mg to 90 mg, 1 mg to 45 mg or 1 mg to 18 mg for the treatment of hair loss, wherein the treatment results in a percentage of hair regrowth ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

21. Intradermal, topical or electrophoretic administration of Nivolumab at a dose ranging from 1 mg to 180 mg, 1 mg to 90 mg, 1 mg to 45 mg or 1 mg to 18 mg for the treatment of skin fine lines (e.g., reduction of skin fine lines), wherein the treatment results in a percentage of skin fine line reduction ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

22. Intradermal, topical or electrophoretic administration of Nivolumab at a dose ranging from 1 mg to 180 mg, 1 mg to 90 mg, 1 mg to 45 mg or 1 mg to 18 mg for the treatment of skin laxity, wherein the treatment results in a percentage reduction of skin laxity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

23. Intradermal, topical or electrophoretic administration of Nivolumab at a dose ranging from 1 mg to 180 mg, 1 mg to 90 mg, 1 mg to 45 mg or 1 mg to 18 mg for the treatment of skin sun spots, wherein the treatment results in a percentage reduction of the number of skin sun spots ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

24. Intradermal, topical or electrophoretic administration of Nivolumab at a dose ranging from 1 mg to 180 mg, 1 mg to 90 mg, 1 mg to 45 mg or 1 mg to 18 mg for the treatment of skin color, wherein the treatment results in a percentage restoration of natural skin color ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

25. Intradermal, topical or electrophoretic administration of Nivolumab at a dose ranging from 1 mg to 180 mg, 1 mg to 90 mg, 1 mg to 45 mg or 1 mg to 18 mg for the treatment of skin elasticity, wherein the treatment results in a percentage increase of skin elasticity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

26. Intradermal, topical or electrophoretic administration of Nivolumab at a dose ranging from 1 mg to 180 mg, 1 mg to 90 mg, 1 mg to 45 mg or 1 mg to 18 mg for the treatment of skin pores, wherein the treatment results in a percentage decrease of skin pore size ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

27. Intradermal, topical or electrophoretic administration of Nivolumab at a dose ranging from 1 mg to 180 mg, 1 mg to 90 mg, 1 mg to 45 mg or 1 mg to 18 mg for the treatment of skin hydration, wherein the treatment results in a percentage increase of skin hydration ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

28. Intradermal, topical or electrophoretic administration of Cimiplimab at a dose ranging from 1 mg to 175 mg, 1 mg to 85 mg, 1 mg to 40 mg or 1 mg to 18 mg for the treatment of hair pigmentation loss, where the treatment results in a percentage of hair repigmentation ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

29. Intradermal, topical or electrophoretic administration of Cimiplimab at a dose ranging from 1 mg to 175 mg, 1 mg to 85 mg, 1 mg to 40 mg or 1 mg to 18 mg for the treatment of hair loss, wherein the treatment results in a percentage of hair regrowth ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

30. Intradermal, topical or electrophoretic administration of Cimiplimab at a dose ranging from 1 mg to 175 mg, 1 mg to 85 mg, 1 mg to 40 mg or 1 mg to 18 mg for the treatment of skin fine lines (e.g., reduction of skin fine lines), wherein the treatment results in a percentage of skin fine line reduction ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

31. Intradermal, topical or electrophoretic administration of Cimiplimab at a dose ranging from 1 mg to 175 mg, 1 mg to 85 mg, 1 mg to 40 mg or 1 mg to 18 mg for the treatment of skin laxity, wherein the treatment results in a percentage reduction of skin laxity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

32. Intradermal, topical or electrophoretic administration of Cimiplimab at a dose ranging from 1 mg to 175 mg, 1 mg to 85 mg, 1 mg to 40 mg or 1 mg to 18 mg for the treatment of skin sun spots, wherein the treatment results in a percentage reduction of the number of skin sun spots ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

33. Intradermal, topical or electrophoretic administration of Cimiplimab at a dose ranging from 1 mg to 175 mg, 1 mg to 85 mg, 1 mg to 40 mg or 1 mg to 18 mg for the treatment of skin color, wherein the treatment results in a percentage restoration of natural skin color ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

34. Intradermal, topical or electrophoretic administration of Cimiplimab at a dose ranging from 1 mg to 175 mg, 1 mg to 85 mg, 1 mg to 40 mg or 1 mg to 18 mg for the treatment of skin elasticity, wherein the treatment results in a percentage increase of skin elasticity ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

35. Intradermal, topical or electrophoretic administration of Cimiplimab at a dose ranging from 1 mg to 175 mg, 1 mg to 85 mg, 1 mg to 40 mg or 1 mg to 18 mg for the treatment of skin pores, wherein the treatment results in a percentage decrease of skin pore size ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

36. Intradermal, topical or electrophoretic administration of Cimiplimab at a dose ranging from 1 mg to 175 mg, 1 mg to 85 mg, 1 mg to 40 mg or 1 mg to 18 mg for the treatment of skin hydration, wherein the treatment results in a percentage increase of skin hydration ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

37. Intradermal, topical or electrophoretic administration of Atezolizumab at a dose ranging from 1 mg to 600 mg, 1 mg to 300 mg, 1 mg to 150 mg or 1 mg to 60 mg for the treatment of hair pigmentation loss, where the treatment results in a percentage of hair repigmentation ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

38. Intradermal, topical or electrophoretic administration of Atezolizumab at a dose ranging from 1 mg to 600 mg, 1 mg to 300 mg, 1 mg to 150 mg or 1 mg to 60 mg for the treatment of hair loss, wherein the treatment results in a percentage of hair regrowth ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

39. Intradermal, topical or electrophoretic administration of Atezolizumab at a dose ranging from 1 mg to 600 mg, 1 mg to 300 mg, 1 mg to 150 mg or 1 mg to 60 mg for the treatment of skin fine lines (e.g., reduction of skin fine lines), wherein the treatment results in a percentage of skin fine line reduction ranging from 1 percent to 100 percent, 1 percent to 50 I percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

40. Intradermal, topical or electrophoretic administration of Atezolizumab at a dose ranging from 1 mg to 600 mg, 1 mg to 300 mg, 1 mg to 150 mg or 1 mg to 60 mg for the treatment of skin laxity, wherein the treatment results in a percentage reduction of skin laxity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

41. Intradermal, topical or electrophoretic administration of Atezolizumab at a dose ranging from 1 mg to 600 mg, 1 mg to 300 mg, 1 mg to 150 mg or 1 mg to 60 mg for the treatment of skin sun spots, wherein the treatment results in a percentage reduction of the number of skin sun spots ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

42. Intradermal, topical or electrophoretic administration of Atezolizumab at a dose ranging from 1 mg to 600 mg, 1 mg to 300 mg, 1 mg to 150 mg or 1 mg to 60 mg for the treatment of skin color, wherein the treatment results in a percentage restoration of natural skin color ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

43. Intradermal, topical or electrophoretic administration of Atezolizumab at a dose ranging from 1 mg to 600 mg, 1 mg to 300 mg, 1 mg to 150 mg or 1 mg to 60 mg for the treatment of skin elasticity, wherein the treatment results in a percentage increase of skin elasticity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

44. Intradermal, topical or electrophoretic administration of Atezolizumab at a dose ranging from 1 mg to 600 mg, 1 mg to 300 mg, 1 mg to 150 mg or 1 mg to 60 mg for the treatment of skin pores, wherein the treatment results in a percentage decrease of skin pore size ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

45. Intradermal, topical or electrophoretic administration of Atezolizumab at a dose ranging from 1 mg to 600 mg, 1 mg to 300 mg, 1 mg to 150 mg or 1 mg to 60 mg for the treatment of skin hydration, wherein the treatment results in a percentage increase of skin hydration ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

46. Intradermal, topical or electrophoretic administration of Avelumab at a dose ranging from 1 mg to 400 mg, 1 mg to 200 mg, 1 mg to 100 mg or 1 mg to 40 mg for the treatment of hair pigmentation loss, where the treatment results in a percentage of hair repigmentation ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

47. Intradermal, topical or electrophoretic administration of Avelumab at a dose ranging from 1 mg to 400 mg, 1 mg to 200 mg, 1 mg to 100 mg or 1 mg to 40 mg for the treatment of hair loss, wherein the treatment results in a percentage of hair regrowth ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

48. Intradermal, topical or electrophoretic administration of Avelumab at a dose ranging from 1 mg to 400 mg, 1 mg to 200 mg, 1 mg to 100 mg or 1 mg to 40 mg for the treatment of skin fine lines (e.g., reduction of skin fine lines), wherein the treatment results in a percentage of skin fine line reduction ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

49. Intradermal, topical or electrophoretic administration of Avelumab at a dose ranging from 1 mg to 400 mg, 1 mg to 200 mg, 1 mg to 100 mg or 1 mg to 40 mg for the treatment of skin laxity, wherein the treatment results in a percentage reduction of skin laxity ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

50. Intradermal, topical or electrophoretic administration of Avelumab at a dose ranging from 1 mg to 400 mg, 1 mg to 200 mg, 1 mg to 100 mg or 1 mg to 40 mg for the treatment of skin sun spots, wherein the treatment results in a percentage reduction of the number of skin sun spots ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

51. Intradermal, topical or electrophoretic administration of Avelumab at a dose ranging from 1 mg to 400 mg, 1 mg to 200 mg, 1 mg to 100 mg or 1 mg to 40 mg for the treatment of skin color, wherein the treatment results in a percentage restoration of natural skin color ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

52. Intradermal, topical or electrophoretic administration of Avelumab at a dose ranging from 1 mg to 400 mg, 1 mg to 200 mg, 1 mg to 100 mg or 1 mg to 40 mg for the treatment of skin elasticity, wherein the treatment results in a percentage increase of skin elasticity ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

53. Intradermal, topical or electrophoretic administration of Avelumab at a dose ranging from 1 mg to 400 mg, 1 mg to 200 mg, 1 mg to 100 mg or 1 mg to 40 mg for the treatment of skin pores, wherein the treatment results in a percentage decrease of skin pore size ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

54. Intradermal, topical or electrophoretic administration of Avelumab at a dose ranging from 1 mg to 400 mg, 1 mg to 200 mg, 1 mg to 100 mg or 1 mg to 40 mg for the treatment of skin hydration, wherein the treatment results in a percentage increase of skin hydration ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

55. Intradermal, topical or electrophoretic administration of Durvalumab at a dose ranging from 1 mg to 750 mg, 1 mg to 375 mg, 1 mg to 180 mg or 1 mg to 75 mg for the treatment of hair pigmentation loss, where the treatment results in a percentage of hair repigmentation ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

56. Intradermal, topical or electrophoretic administration of Durvalumab at a dose ranging from 1 mg to 750 mg, 1 mg to 375 mg, 1 mg to 180 mg or 1 mg to 75 mg for the treatment of hair loss, wherein the treatment results in a percentage of hair regrowth ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

57. Intradermal, topical or electrophoretic administration of Durvalumab at a dose ranging from 1 mg to 750 mg, 1 mg to 375 mg, 1 mg to 180 mg or 1 mg to 75 mg for the treatment of skin fine lines (e.g., reduction of skin fine lines), wherein the treatment results in a percentage of skin fine line reduction ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

58. Intradermal, topical or electrophoretic administration of Durvalumab at a dose ranging from 1 mg to 750 mg, 1 mg to 375 mg, 1 mg to 180 mg or 1 mg to 75 mg for the treatment of skin laxity, wherein the treatment results in a percentage reduction of skin laxity ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

59. Intradermal, topical or electrophoretic administration of Durvalumab at a dose ranging from 1 mg to 750 mg, 1 mg to 375 mg, 1 mg to 180 mg or 1 mg to 75 mg for the treatment of skin sun spots, wherein the treatment results in a percentage reduction of the number of skin sun spots ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

60. Intradermal, topical or electrophoretic administration of Durvalumab at a dose ranging from 1 mg to 750 mg, 1 mg to 375 mg, 1 mg to 180 mg or 1 mg to 75 mg for the treatment of skin color, wherein the treatment results in a percentage restoration of natural skin color ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

61. Intradermal, topical or electrophoretic administration of Durvalumab at a dose ranging from 1 mg to 750 mg, 1 mg to 375 mg, 1 mg to 180 mg or 1 mg to 75 mg for the treatment of skin elasticity, wherein the treatment results in a percentage increase of skin elasticity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

62. Intradermal, topical or electrophoretic administration of Durvalumab at a dose ranging from 1 mg to 750 mg, 1 mg to 375 mg, 1 mg to 180 mg or 1 mg to 75 mg for the treatment of skin pores, wherein the treatment results in a percentage decrease of skin pore size ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

63. Intradermal, topical or electrophoretic administration of Durvalumab at a dose ranging from 1 mg to 750 mg, 1 mg to 375 mg, 1 mg to 180 mg or 1 mg to 75 mg for the treatment of skin hydration, wherein the treatment results in a percentage increase of skin hydration ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

64. Intradermal, topical or electrophoretic administration of Ipilimumab at a dose ranging from 1 mg to 100 mg, 1 mg to 50 mg, 1 mg to 25 mg or 1 mg to 10 mg for the treatment of hair pigmentation loss, where the treatment results in a percentage of hair repigmentation ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

65. Intradermal, topical or electrophoretic administration of Ipilimumab at a dose ranging from 1 mg to 100 mg, 1 mg to 50 mg, 1 mg to 25 mg or 1 mg to 10 mg for the treatment of hair loss, wherein the treatment results in a percentage of hair regrowth ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

66. Intradermal, topical or electrophoretic administration of Ipilimumab at a dose ranging from 1 mg to 100 mg, 1 mg to 50 mg, 1 mg to 25 mg or 1 mg to 10 mg for the treatment of skin fine lines (e.g., reduction of skin fine lines), wherein the treatment results in a percentage of skin fine line reduction ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

67. Intradermal, topical or electrophoretic administration of Ipilimumab at a dose ranging from 1 mg to 100 mg, 1 mg to 50 mg, 1 mg to 25 mg or 1 mg to 10 mg for the treatment of skin laxity, wherein the treatment results in a percentage reduction of skin laxity ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

68. Intradermal, topical or electrophoretic administration of Ipilimumab at a dose ranging from 1 mg to 100 mg, 1 mg to 50 mg, 1 mg to 25 mg or 1 mg to 10 mg for the treatment of skin sun spots, wherein the treatment results in a percentage reduction of the number of skin sun spots ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

69. Intradermal, topical or electrophoretic administration of Ipilimumab at a dose ranging from 1 mg to 100 mg, 1 mg to 50 mg, 1 mg to 25 mg or 1 mg to 10 mg for the treatment of skin color, wherein the treatment results in a percentage restoration of natural skin color ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

70. Intradermal, topical or electrophoretic administration of Ipilimumab at a dose ranging from 1 mg to 100 mg, 1 mg to 50 mg, 1 mg to 25 mg or 1 mg to 10 mg for the treatment of skin elasticity, wherein the treatment results in a percentage increase of skin elasticity ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

71. Intradermal, topical or electrophoretic administration of Ipilimumab at a dose ranging from 1 mg to 100 mg, 1 mg to 50 mg, 1 mg to 25 mg or 1 mg to 10 mg for the treatment of skin pores, wherein the treatment results in a percentage decrease of skin pore size ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

72. Intradermal, topical or electrophoretic administration of Ipilimumab at a dose ranging from 1 mg to 100 mg, 1 mg to 50 mg, 1 mg to 25 mg or 1 mg to 10 mg for the treatment of skin hydration, wherein the treatment results in a percentage increase of skin hydration ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

73. Intradermal, topical or electrophoretic administration of Tremelimumab at a dose ranging from 1 mg to 375 mg, 1 mg to 180 mg, 1 mg to 90 mg or 1 mg to 35 mg for the treatment of hair pigmentation loss, where the treatment results in a percentage of hair repigmentation ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

74. Intradermal, topical or electrophoretic administration of Tremelimumab at a dose ranging from 1 mg to 375 mg, 1 mg to 180 mg, 1 mg to 90 mg or 1 mg to 35 mg for the treatment of hair loss, wherein the treatment results in a percentage of hair regrowth ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

75. Intradermal, topical or electrophoretic administration of Tremelimumab at a dose ranging from 1 mg to 375 mg, 1 mg to 180 mg, 1 mg to 90 mg or 1 mg to 35 mg for the treatment of skin fine lines (e.g., reduction of skin fine lines), wherein the treatment results in a percentage of skin fine line reduction ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

76. Intradermal, topical or electrophoretic administration of Tremelimumab at a dose ranging from 1 mg to 375 mg, 1 mg to 180 mg, 1 mg to 90 mg or 1 mg to 35 mg for the treatment of skin laxity, wherein the treatment results in a percentage reduction of skin laxity ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

77. Intradermal, topical or electrophoretic administration of Tremelimumab at a dose ranging from 1 mg to 375 mg, 1 mg to 180 mg, 1 mg to 90 mg or 1 mg to 35 mg for the treatment of skin sun spots, wherein the treatment results in a percentage reduction of the number of skin sun spots ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

78. Intradermal, topical or electrophoretic administration of Tremelimumab at a dose ranging from 1 mg to 375 mg, 1 mg to 180 mg, 1 mg to 90 mg or 1 mg to 35 mg for the treatment of skin color, wherein the treatment results in a percentage restoration of natural skin color ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

79. Intradermal, topical or electrophoretic administration of Tremelimumab at a dose ranging from 1 mg to 375 mg, 1 mg to 180 mg, 1 mg to 90 mg or 1 mg to 35 mg for the treatment of skin elasticity, wherein the treatment results in a percentage increase of skin elasticity ranging from 1 percent to 100 percent, from 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

80. Intradermal, topical or electrophoretic administration of Tremelimumab at a dose ranging from 1 mg to 375 mg, 1 mg to 180 mg, 1 mg to 90 mg or 1 mg to 35 mg for the treatment of skin pores, wherein the treatment results in a percentage decrease of skin pore size ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

81. Intradermal, topical or electrophoretic administration of Tremelimumab at a dose ranging from 1 mg to 375 mg, 1 mg to 180 mg, 1 mg to 90 mg or 1 mg to 35 mg for the treatment of skin hydration, wherein the treatment results in a percentage increase of skin hydration ranging from 1 percent to 100 percent, 1 percent to 50 percent, from 1 percent to 25 percent, from 1 percent to 10 percent or from 1 percent to 5 percent after one to ten treatments (e.g., 2, 3, 4, 5, 6, 7) within six months after the last treatment (e.g., three weeks or three months after the treatment).

Various Immune Checkpoint Inhibitors - including Pembrolizumab, Nivolumab, Cemiplimab, Atezolizumab, Avelumab, Durvalab, Ipilimumab and Tremelimumab - are commercially available from a variety of sources. One such source is SelleckChem.com. Others can be obtained using published literature methods. For example, see, Marin-Acevedo et al. “Next generation of immune checkpoint inhibitors and beyond” Journal of Hematology & Oncology volume 14, Article number: 45 (2021), and references therein.

### EXAMPLES

An Immune Checkpoint Inhibitor is diluted with solvent (e.g., Normal Saline) to a desired concentration to provide a solution (e.g., 1-5 mg/mL).

Approximately 0.5 mL of the solution is drawn in a syringe. A portion of the solution, typically around 0.1 mL, is applied to a small area on a patient’s skin (e.g., approximately 2 cm x 2 cm depending upon area). A motorized microneedle pen with 12 needles, or a microneedle roller, is used at highest speed with penetration depth dependent upon location (e.g., 1-2 mm). (If a single-needle syringe is used instead of a microneedling device, the medication is administered intradermally with a syringe.) The Immune Checkpoint Inhibitor solution is applied to the patient’s skin using the microneedling device approximately 4x in perpendicular directions. Application is continued until all desired areas are covered. The patient should not wash her skin, go into the sun or apply skin products to the treated area for approximately 24 hours. This procedure may be repeated, for example, every three weeks, until the desired result is obtained.

A number of experiments were run. For these experiments, pembrolizumab (25 mg/mL) was diluted with NS to 2.5 mg/mL. 0.5 cc of the diluted pembrolizumab was placed in a syringe. The patient’s face was wiped clean with witch hazel. The 0.5 cc of diluted pembrolizumab was applied to the desired skin area using a microneedle until all desired areas were covered. It was then rubbed into the area. The solution was applied to the desired skin area every month.

A dermatology photo robot (Visia Skin Analysis Imaging System from Canfield) was used to calculate scores for different skin parameters at the start (baseline, before any pembrolizumab application), at one month of pembrolizumab applications and at four months of pembrolizumab applications. Skin parameters that were evaluated include: skin spots; skin wrinkles; skin texture; skin pore size; UV skin spots; brown skin spots; red skin spots; and porphyrin skin level. Each parameter measurement was taken at three different locations on the patient - front, left and right. The following values were obtained, where a higher value indicated a worse outcome (e.g., more skin spots, more skin wrinkles, worse skin texture, larger pore size, more UV skin spots, more brown skin spots, more red skin spots, higher porphyrin skin level) and a lower value indicated a better outcome (e.g., fewer skin spots, fewer skin wrinkles, better skin texture, smaller pore size, fewer UV skin spots, fewer brown skin spots, fewer red skin spots, lower porphyrin skin level) and calculated measurement errors are included after each presented value:

Skin spots score: Baseline - Front 37.66667 (error 0.339618), Left 41 (error 0.588235), Right 38 (error 0.588235); One Month - Front 36.33333 (error 0.898544), Left 37.33333 (error 0.898544), Right 35.66667 (error 0.898544); Four Months - Front 30 (error 0.588235); Left 32.66667 (error 1.358471); Right - 32 (error 1.556324).

Skin wrinkles score: Baseline - Front 11.66667 (error 0.339618), Left 33.66667 (error 1.224509), Right 28.33333 (error 0.898544); One Month - Front 11.66667 (error 0.898544), Left - 25.33333 (error 1.698089), Right - 35 (error 1.176471); Fourth Months - Front 17.33333 (error 2.377325), Left - 29.66667 (error 0.679236), Right - 32 (error 1.018853).

Skin texture score: Baseline - Front 8.733333 (error 0.135847), Left - 17.66667 (0.679236), Right - 21 (error 0); One Month - Front 6.033333 (error 0.296072), Left 10.66667 (error 0.339618), Right 18.66667 (error 0.339618); Four Months - Front 4.933333 (error 0.148036), Left - 9.3 (error 0.35781), Right - 9.633333 (error 0.277989).

Skin pores score: Baseline - Front 10.83333 (error 0.169809), Left 12 (error 0), Right 9.566667 (error 0.222702); One Month - Front 9.766667 (error 0.237732), Left 11.66667 (error 0.339618), Right 9.5 (error 0.212091); Four Months - Front 10 (error 0), Left 11.33333 (error 0.339618), Right - 9.233333 (error 0.301859).

Skin UV spots score: Baseline - Front 16.66667 (error 0.339618), Left 20.66667 (error 0.339618), Right 21.33333 (error 0.679236); One Month - Front 16.33333 (error 0.339618), Left 20.66667 (error 0.339618), Right 21 (error 0.588235); Four Months - Front 17.33333 (error 0.679236), Left 23.66667 (error 0.339618), Right 22.33333 (error 0.339618).

Skin brown spots score: Baseline - Front 18.66667 (error 0.339618), Left 21.33333 (error 0.679236), Right 21 (error 0); One Month - Front 19 (error 0.588235), Left 22 (error 0), Right 22 (error 0); Four Months - Front 17.66667 (error 0.339618), Left 20.33333 (error 0.339618), Right 19 (error 0).

Skin red spots score: Baseline - 15.33333 (error 0.339618), Left 17.33333 (error 0.339618), Right 16.66667 (error 0.339618); One Month - Front 12 (error 0), Left 14 (error 0), Right 13 (error 0); Four Months - Front 12.33333 (error 0.339618), Left 13 (error 0), Right 14.66667 (error 0.339618).

Skin porphyrins score: Baseline - 22.33333 (error 0.339618), Left 18.66667 (error 0.898544), Right 22 (0.588235); One Month - Front 19.66667 (error 0.339618), Left 16.66667 (error 0.339618), Right 17.66667 (error 0.679236); Four Months - Front 22 (error 0), Left 21 (error 0.588235), Right 20.66667 (error 0.339618).

The same protocol was used to test the ability of pembrolizumab to ameliorate loss of hair pigmentation. Pembrolizumab (25 mg/mL) was diluted with NS to 2.5 mg/mL. 0.5 cc of the diluted pembrolizumab was placed in a syringe. The patient’s face was wiped clean with witch hazel. The 0.5 cc of diluted pembrolizumab was applied to the desired skin area, which included graying beard stubble, using a microneedle until all desired areas were covered. It was then rubbed into the area. The solution was applied to the desired skin area every month. After three months the beard stubble was visibly darkened.

1. A method of treating an effect of aging, wherein the effect of aging is selected from a group consisting of loss of hair pigmentation, hair loss, increase in fine lines, increase in skin laxity, increase of skin sun spots, loss of natural skin color, loss of skin elasticity, increase in skin pore size, rosacea, actinic keratosis and loss of skin hydration, wherein the method comprises the steps of:
obtaining or preparing an Immune Checkpoint Inhibitor formulation, wherein the formulation comprises an Immune Checkpoint Inhibitor and water, and wherein the concentration of the Immune Checkpoint Inhibitor in the formation ranges from 1 mg/mL to 60 mg/mL;
administering a portion of the formula intradermally, topically or electrophoretically to a patient experiencing an aging effect to a skin area on the patient, wherein the amount of Immune Checkpoint Inhibitor administered in the formulation ranges from 1 mg to 100 mg,
wherein an aging effect is lessened by a percentage ranging from 1 percent to 100 percent within six months after intradermal administration.

2. The method according to claim 1, wherein the Immune Checkpoint Inhibitor is Pembrolizumab, and wherein the amount of Pembrolizumb administered ranges from 1 mg to 100 mg.

3. The method according to claim 1, wherein the Immune Checkpoint Inhibitor is Nivolumab, and wherein the amount of Nivolumab administered ranges from 1 mg to 180 mg.

4. The method according to claim 1, wherein the Immune Checkpoint Inhibitor is Cimiplimab, and wherein the amount of Cimiplimab administered ranges from 1 mg to 175 mg.

5. The method according to claim 1, wherein the Immune Checkpoint Inhibitor is Atezolizumab, and wherein the amount of Atezolizumab administered ranges from 1 mg to 600 mg.

6. The method according to claim 1, wherein the Immune Checkpoint Inhibitor is Avelumab, and wherein the amount of Avelumab administered ranges from 1 mg to 400 mg.

7. The method according to claim 1, wherein the Immune Checkpoint Inhibitor is Durvalumab, and wherein the amount of Durvalumab administered ranges from 1 mg to 750 mg.

8. The method according to claim 1, wherein the Immune Checkpoint Inhibitor is Ipilimumab, and wherein the amount of Ipilimumab administered ranges from 1 mg to 100 mg.

9. The method according to claim 1, wherein the Immune Checkpoint Inhibitor is Tremelimumab, and wherein the amount of Tremelimumab administered ranges from 1 mg to 375 mg.

10. The method according to claim 1, wherein the Immune Checkpoint inhibitor is selected from a group consisting of: LAG525 (IMP701); REGN3767 (R3767); BI 754,091; Tebotelimab (MGD013); FS118; MBG453; Sym023; TSR-022; MGC018; FPA150; EOS100850; AB928; CPI-006; Monalizumab; COM701; Defactinib; PF-04136309; MSC-1; Hu5F9-G4 (5F9); ALX148; TTI-662; RRx-001; Lacnotuzumab (MCS110); Emactuzumab (RG7155); Pexidartinib (PLX3397); CAN04; Canakinumab (ACZ885); BMS-986253; Pepinemab (VX15/2503); Trebananib; FP-1305.
